(19) United States
(12) Patent Application Publication
(10) Pub. No.: US 2006/0013409 A1
(43) Pub. Date: Jan. 19, 2006

(54) Microphone-array processing to generate directional cues in an audio signal

(75) Inventor: Joseph G. Desloge, Brookline, MA (US)

Correspondence Address: WILMER CUTLER PICKERING HALE AND DORR LLP, 60 STATE STREET, BOSTON, MA, 02109, US

(73) Assignee: Sensimetrics Corporation, Somerville, MA (US)

(21) Appl. No.: 10/893,188

(22) Filed: Jul. 16, 2004

(51) Int. Cl. A61F 11/06; H04R 1/10

(52) U.S. Cl. 381/72; 381/71.6; 381/74

(57) ABSTRACT

A hear-through hearing protective device includes at least two microphones, simulated-pinna filters, and a processing circuit. Elevation dependent directional cues are introduced by the processing performed by the simulated-pinna filters. Introduced directional cues are designed to allow the wearer to identify the source location of an acoustic signal.

### BACKGROUND

Hearing protectors are designed to shield the user from loud external sounds. They accomplish this goal by blocking the ear and reducing the intensity of external sounds reaching the ear canal. Such protection helps to safeguard the hearing of people who are routinely exposed to high-level noise.

One problem with typical hearing protectors is that they reduce all sounds. While protecting the user from loud, damaging sounds, they also reduce the user's ability to hear important sounds at normal intensity. The loss of normal-level auditory stimuli isolates the wearer from the surrounding environment, thereby delaying or even preventing the wearer's reaction to low-level sounds (e.g., the wearer cannot hear spoken communication from people nearby). Such isolation can cause the wearer to remove the hearing protection, which then leaves the wearer vulnerable to unexpected loud sounds.

In order to address this problem, many hearing protection systems offer “hear-through” capabilities. In such systems, microphones are located near the two ears. These microphone signals are automatically controlled for sound level and fed electronically through the hearing protection and presented at the ear canal. By automatically controlling the signal level, these hear-through systems allow normal-volume sounds to reach the ear unchanged while attenuating loud-volume sounds to prevent hearing damage.

Current hear-through systems degrade the user's ability to localize sounds. Humans estimate where sounds come from by sensing acoustic characteristics of the signals received at the ears. Some of these characteristics are related to differences between the signals at the two ears (interaural differences), and others are related to the spectral shaping imposed by the head and pinna (outer ear) through head-related transfer functions (HRTFs). Signals from the pick-up microphones in current hear-through systems are filtered by HRTFs that differ substantially from the natural ones that characterize a person's open ears. Without appropriate HRTFs, the user's ability to localize sound sources in space degrades.

Current hear-through systems preserve some but not all of the important HRTF cues. By simply locating the microphones near the two ears, the natural interaural (level and time difference) cues are approximately retained. The pinna-related HRTF cues, however, are generally lost. Specifically, when the hear-through protector consists of muffs that completely cover the external ear, microphone signals taken from outside the muff can lose all cues provided by the pinnae. When the hearing protection consists of earplugs, the plugs often fill the conchae and microphone signals taken from outside the plug lose important concha-reflection cues. The concha is the largest and deepest concavity of the external ear. This loss of pinna HRTF cues reduces the listener's ability to determine the elevation and the front-back orientation of a sound source.

### SUMMARY

Systems and methods are described for re-introducing some pinna cues into hear-through hearing-protection systems, such as muffs or ear plugs. Two or more microphones are provided at each ear to create spectral features (e.g., notches) that depend on the location of the source to mimic those generated naturally by the user's pinnae. As with the currently available hear-through systems, the interaural HRTF cues are approximately preserved by placing the microphone clusters near the two ears.

In certain embodiments, left and right omnidirectional pick-up microphones are replaced with left and right clusters of microphones with the goal of generating a hear-through hearing protection system that preserves pinna-dependent localization cues. The system can specifically apply the location-dependent frequency-response capabilities of multi-microphone systems to the task of reproducing human spectral localization cues. The methods described herein are generally referred to as simulated-pinna processing.

According to certain embodiments, a device for mimicking directional cues of an acoustic signal includes a circumaural muff with first and second spatially-separated microphones outside a muff (or other hearing protection device) for receiving the acoustic signal and communicates respective first and second signals. The first signal is substantially similar to the second signal but shifted in time relative to the second signal due to the displacement between the microphones. A circuit can process and combine the first and second signals in accordance with the time shift, the lateral displacement of the microphones, and the predetermined direction. An amplifying circuit can be provided to amplify the resultant processed signal. A driver can be provided inside the hearing protection device for receiving the amplified signal electromechanically and transmitting a second acoustic signal into the interior of the hearing protection device.

A processor can receive the electrical signals and process the signals in accordance with a source-location dependent frequency notch in the frequency spectra. The processor combines the electrical signals and introduces directional cues into the combination of electrical signals. The device can further include an amplifier for amplifying the processed electrical signal. A driver is provided on the inside of the hearing protection device for receiving the combination of electrical signals. The driver transmits a second acoustic signal into the interior of the hearing protection device. The frequency notch is a result of a destructive interference caused by the propagation delay between the first and second acoustic microphone signals and the signal processing used to combine these signals.

Such hear-through hearing protectors can be used for industrial and military purposes, target shooting, hunting, or for other applications. Other features and advantages will be appreciated to one skilled in the art.

### BRIEF DESCRIPTION OF THE DRAWING

FIG. 1 is a perspective view of a muff with a microphone cluster mounted on outside of the muff.

FIG. 2 is a perspective view of a muff and microphone system as worn.

FIG. 3 is a block diagram of signal processing to introduce HRTF cues into an output signal.

FIGS. 4A and 4B are waveforms showing received left-ear source spectra for sources from 0 degrees azimuth and 0 and 60 degrees elevation taken from measured KEMAR HRTFs (top panel) and from simulated-pinna processing (bottom panel).

FIG. 5 is a block diagram showing a simulated-pinna processing operation.

### DETAILED DESCRIPTION

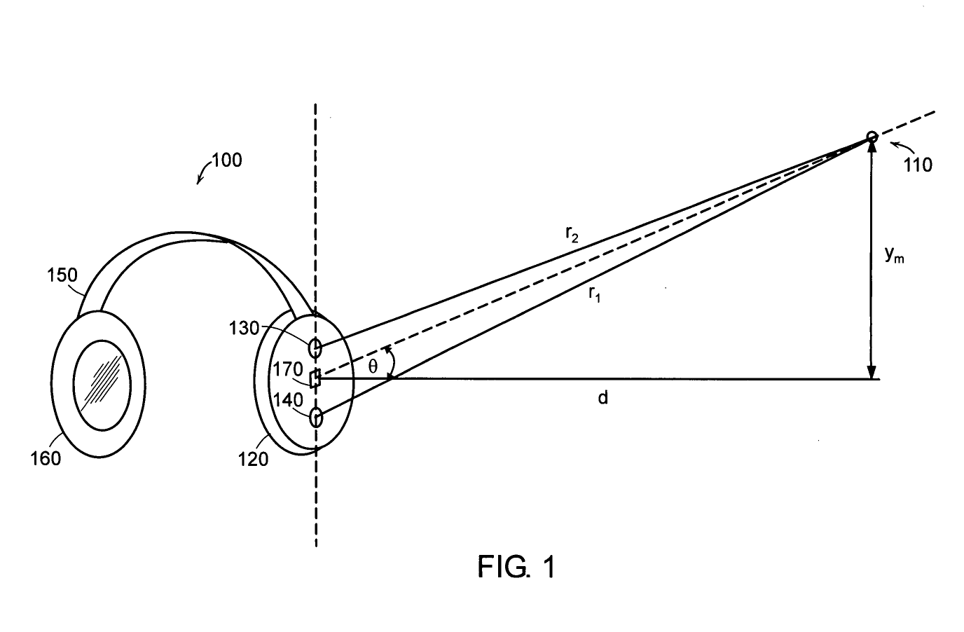

Referring to FIG. 1, a hear-through hearing protective device 100 is shown with an acoustic source 110. Hear-through hearing protective device 100 includes a first circumaural ear muff 120, a headband 150, and second circumaural ear muff 160. Headband 150 holds ear muffs 120, 160 to the user's head (not shown). Muff 160 can be substantially similar (i.e., a mirror image) to ear muff 120 or it can be different. While described in the context of a muff, other hearing protection devices could be used, such as ear plugs.

Ear muff 120 includes a first microphone 130, a second microphone 140 spaced from first microphone 130, and a processing circuit 170. These microphones can be located physically on the outside of the muff, or they can be outside the muff but not necessarily physically on the muff. In one embodiment, processing circuit 170 includes an adder circuit that adds the signals produced from microphones 130 and 140.

A broadband (e.g., 20-20 kHz) acoustic source 110 transmits an acoustic signal with wavelength λᵢ. The acoustic signal is received by microphones 130 and 140, which produce electrical signals representative of the acoustic signal. These electrical signals are substantially similar to one another but shifted in time (or phase) because of the microphone spacing. For instance, the signal produced by 140 is a time-shifted example of the signal received at microphone 130 due to the additional time for the acoustic signal to arrive at microphone 140. The phase difference ΔΦ between the signals produced by microphones 130 and 140 is a function of distances r₁ and r₂ from source 110 to the microphones. The difference between r₁ and r₂ is a function of d, θ (or yₘ), and the spacing between microphones 130 and 140.

An adder circuit can be used to combine the signals produced by microphones 130 and 140. For far-field sources ,where r₁ and r₂ are significantly greater than (e.g., 25 times greater than) the microphone separation, the resultant power of the combined signal is approximately dictated by the following equation:

$$P ∝ \frac{2}{{r}_{1}^{2} + {r}_{2}^{2}} {cos}^{2} ( \frac{π ( {r}_{1} - {r}_{2} )}{{λ}_{i}} ) .$$

Since microphone simulated-pinnae microphone spacings are very small (˜1-2 cm), most sources exhibit ‘significantly large’ enough r₁ and r₂ such that the above relation approximately holds.

For a specific source location (with corresponding r₁ and r₂ ), the combined power exhibits peaks and valleys as a function of λᵢ. The valleys, referred to here as spectral notches, change with source location.

In another embodiment, the processing circuit may include a delay circuit applied to the output of one microphone prior to microphone signal adding. The phase difference ΔΦ between the signals produced by microphones 130 and 140 is a function of the distance between microphones 130 and 140. The delay circuit changes the phase difference ΔΦ between the signals produced by the microphones. As a result, a shift in the location-dependent spectral notches occurs. The delay can be adjusted to shift the spectral notches so that they resemble naturally occurring pinnae-cue spectral notches. Naturally occurring spectral notches can be empirically measured using a KEMAR® manikin (KEMAR is a registered trademark of Knowles Electronics, Inc.).

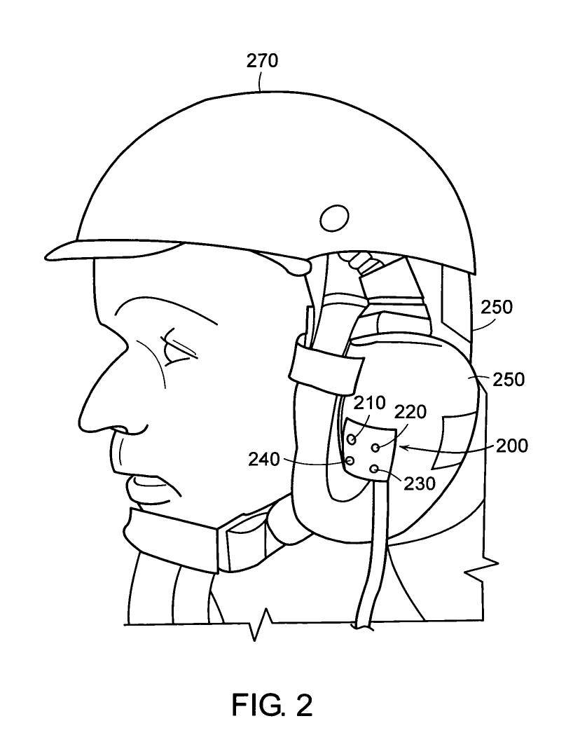

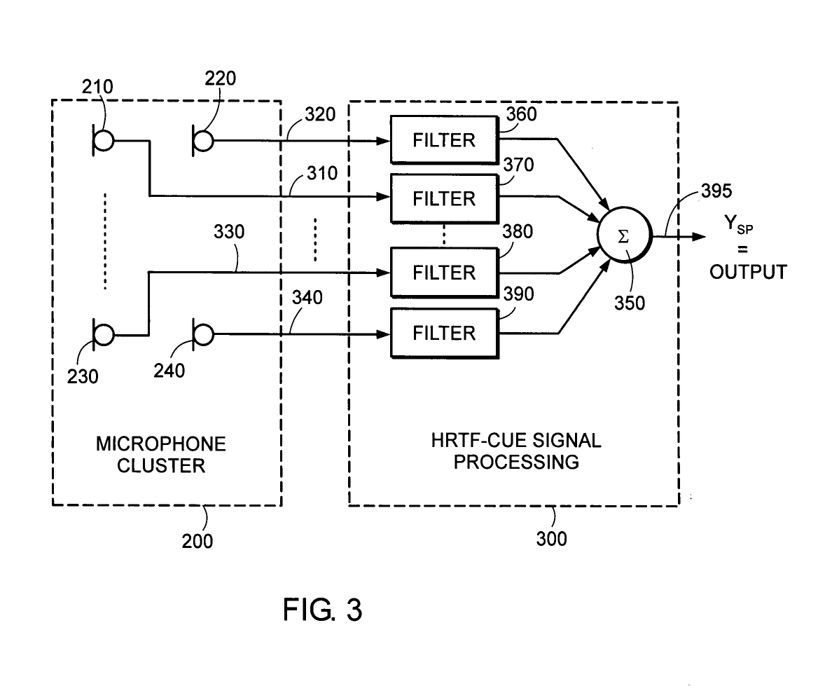

FIGS. 2 and 3 illustrate the operation of what is referred to here as simulated-pinna processing to generate the signal presented to one ear. The signal for the opposite ear is obtained using similar processing on the opposite side of the head. The placement of two simulated-pinna microphone clusters on either side of the head produces relevant interaural source localization cues.

Referring to FIG. 2, a cluster of microphones 200 includes four microphones 210, 220, 230, and 240 in a generally square arrangement on the outside of a hearing muff 250. In an alternate embodiment, a cluster of microphones can be mounted on the outside of an earplug, and in other embodiments, the microphones are arranged in other shapes. Muff 250 can be mounted to a helmet 270 as shown in FIG. 2, or to a headband as shown in FIG. 1. The microphones can be mounted more toward a front area of the muff or at other parts of the muff, or if a muff is incorporated into a helmet, the microphones could be provided on the helmet near the muffs. Ear muff 250 has a head related transfer function (HRTF) cue signal processing circuit (not shown).

FIG. 3 illustrates the processing that is applied to these microphone signals to generate artificial source location cues that mimic the naturally-occurring pinna cues. Specifically, individual microphone signals 310, 320, 330, and 340 that are produced by microphones 210, 220, 230, and 240, respectively, are each passed through a respective signal processing filter 360, 370, 380, and 390. The filters can be analog or digital. The microphone configuration (e.g., arranged in a square), the distance between the microphones, and known HRTF data are used to determine the signal processing that is executed by filters 360, 370, 380, and 390.

The resultant signals processed by filters 360, 370, 380, and 390 are summed by an adder circuit 350 to generate a simulated-pinna output signal 395. The placement of microphones 210, 220, 230, and 240 and the selection of system filters 360, 370, 380, and 390 generate an overall system response that changes with the arrival direction of any given source. By selecting these microphone placement and the filter parameters accordingly, the resulting system can mimic spectral HRTF sound localization cues.

Other microphone configurations can be chosen. Given a particular microphone configuration and a set of microphone filters, the simulated-pinna output signal 395, YSP, for a specific source location is dependent upon the source location, the microphone placement, and the simulated-pinna filters. The desired, naturally-occurring HRTF, represented by variable Y_{HRTF}, is only dependent upon the source location. In the present embodiment, location depends upon azimuth, which refers to the horizontal-plane angle between the source location and straight ahead of the listener, and elevation, which refers to the angle between the source location and a horizontal plane. So, for example, the Y_{HRTF} of (0°,45°) will be different from the Y_{HRTF} of (0°, 0°).

These two signals may be represented as:
Yₛₚ=Yₛₚ(loc,mic,filter) and Y_{HRTF}=Y_{HRTF}(loc),
where loc=(azimuth, elevation) source location,
mic=microphone position,
filter=simulated-pinna filters.

The simulated-pinna system can be designed by selecting the microphone placement and the simulated-pinna filters to minimize the differences between the spectral features of Yₛₚ and Y_{HRTF} that are most useful for source localization.

One general approach to this problem is to use a general error criterion such as:
E(loc,mic,filter)=feature_error[Yₛₚ(loc,mic,filter), Y_{HRTF}(loc)],
where ‘feature_error[A,B]’ measures the error in important spectral source-localization features between the two signals A and B. A location-averaged error may then be formed by averaging over source location:
E_{AVG}(mic,filter)=AVERAGE_{loc}[E(loc,mic,filter)].

Given this average error, it is then possible to select the ‘mic’ and ‘filter’ parameters of the simulated-pinna system to minimize E_{AVG}(mic,filter). This general solution is very flexible in that the function ‘feature_error[A,B]’ can be designed to optimize different combinations of important spectral source localization features such as spectral notches, spectral resonances, etc. This flexibility may require complex simulated-pinna filters that may utilize digital signal processing (DSP) in their implementation. Characteristics of the filters can be determined by simulation, or empirically by using a grid of sources and taking actual measurements.

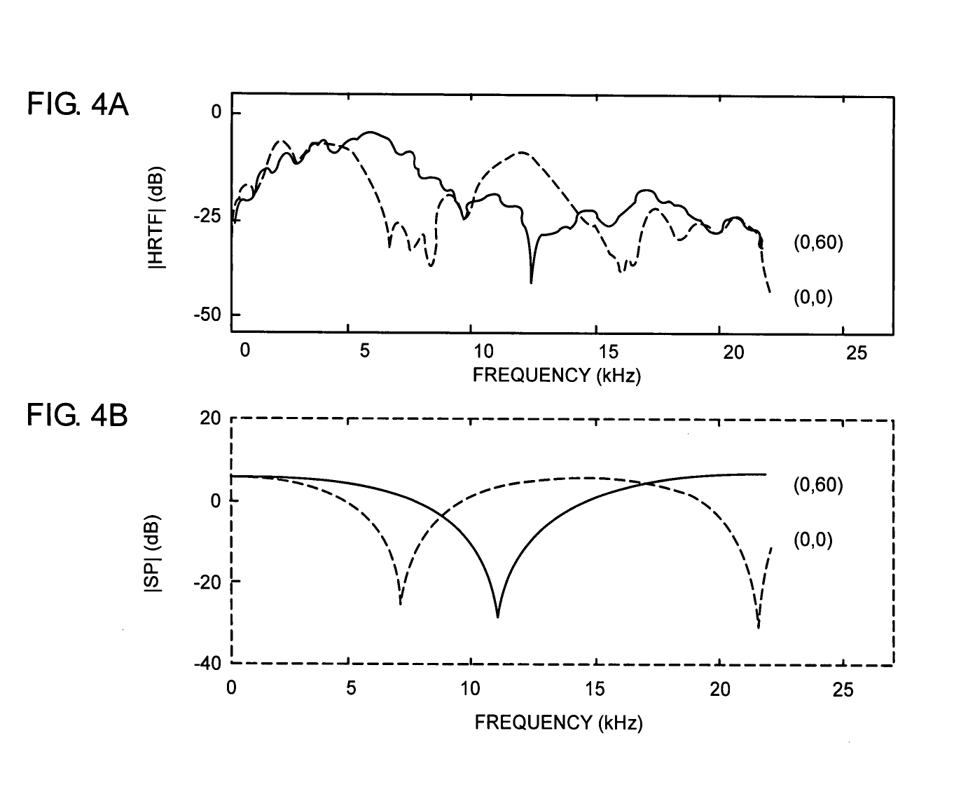

FIGS. 4A and 4B depict bode plots for signals Y_{HRTF} and Yₛₚ, respectively. FIG. 4A illustrates the results of empirically derived data from a KEMAR manikin, with the HRTF shown for two elevations at 0° azimuth. While people can detect sources to the left and right by differences in time and volume, elevation is detected more with spectral cues. The dashed line represents the HRTF for an elevation of 0°. The solid line represents the HRTF for and elevation of 60°. Both lines, measured in dB magnitudes, demonstrate elevation-dependent spectral notches. There is an elevation-dependent notch in this HRTF that occurs at approximately 7.5 kHz for the 0° elevation source and at 12 kHz for the 60° elevation source.

FIG. 4B shows a simplified variation of the simulated-pinna (SP) system that is designed to approximate only a single spectral source localization feature, in this case, elevation-dependent frequency notches. The graph in FIG. 4B exhibits the elevation-dependent notches in the HRTF of FIG. 4A that occurs at approximately 7.5 kHz for the 0° elevation source and at 12 kHz for the 60° elevation source. Spectral notches between the simulated-pinna and the HRTF are matched by adjusting the microphone placement and the parameters of the signal processing filters.

The system thus senses the angle of elevation and provides an elevational spectral notch as a cue to the wearer of the hearing protector.

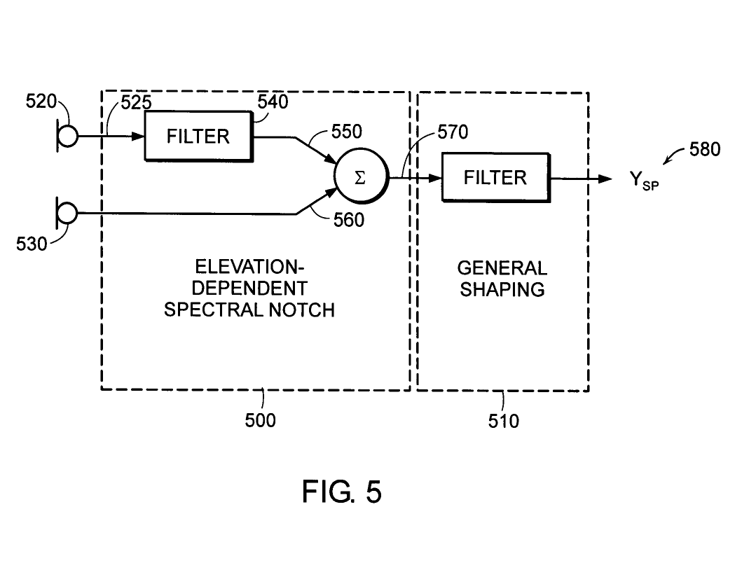

FIG. 5 illustrates another embodiment and shows simulated-pinna processing that can be used to generate this spectral source-localization feature. This system uses two microphones 520, 530 oriented vertically relative to one another.

While different types of processing can be used, in the particular example here, a first stage of processing includes a delay circuit 540 for delaying the top microphone signal 525 and a summer 570 for summing delayed signal 550 with the bottom microphone signal 560. The first stage of processing produces an elevation dependent notch that is controlled through a combination of microphone separation and choice of delay. For example, a microphone separation of 1 cm and a top-microphone delay of 70 μsec leads to the behavior shown in FIG. 4B: the system produces a null at 7.2 kHz for a 0° elevation source and a null at 11.2 kHz for a 60° elevation source. A second stage of processing, such as a single filter 510, can be used to shape the output signal spectrum to approximate location-independent filtering by the pinna (for example, due to pinna resonances).

This simulated-pinna system is somewhat simplified compared to the system in FIG. 3, but it preserves an important spectral source localization feature (elevation-dependent notches), while being simple enough that it can be implemented using low-power analog signal processing.

The invention may be embodied in other specific forms without departing from the spirit or essential characteristics thereof. The present embodiments are therefore to be considered in respects as illustrative and not restrictive, the scope of the invention being indicated by the appended claims rather than by the foregoing description, and all changes which come within the meaning and range of the equivalency of the claims are therefore intended to be embraced therein.

What is claimed is:

1. A system comprising:
a hearing protection device having an inside facing an ear and an outside;
a first microphone at the outside of the hearing protection device, the first microphone receiving an acoustic signal and providing a first signal;
a second microphone at the outside of the hearing protection device and spaced from the first microphone, the second microphone receiving the acoustic signal and providing a second signal;
a circuit for receiving the first and second signals and for processing the first and second signals in accordance with head-related transfer function information to produce a processed signal; and
a driver coupled to the circuit for transmitting to the inside of the hearing protection device the processed signal.

2. The system according to claim 1, wherein the hearing protection device includes a first muff, the system further comprising a second muff similar to the first muff and a headband connecting the first and second muffs, the second muff also having at least first and second microphones, a circuit, and a driver similar to those associated with the first muff.

3. The system according to claim 1, further comprising third and fourth microphones, the four microphones arranged in a rectangular configuration and each coupled to the circuit.

4. The system according to claim 1, wherein the first and second microphones are omnidirectional.

5. The system according to claim 1, wherein the circuit includes a digital signal processor.

6. The system according to claim 1, wherein the first and second microphones are spaced in a vertical direction.

7. The system according to claim 1, wherein the circuit includes a filter for the first signal and an adder for combining the filtered first signal and the second signal.

8. The system according to claim 1, wherein the circuit includes filters.

9. The system of claim 8, wherein the filters are designed to combine the first and second signals to produce an output that simulates directional cues.

10. The system of claim 1, wherein the spectral notch that is introduced provides a wearer of the device with elevational cues to indicate an elevational source of a sound.

11. The system of claim 1, further comprising first and second filters for receiving and filtering the respective first and second signals, the filters filtering in accordance with head-related transfer function information.

12. The system of claim 11, wherein the filters are further configured in accordance with the microphone configuration and a distance between the first and second microphones.

13. The system of claim 11, further comprising third and fourth microphones and third and fourth filters for receiving signals from the respective third and fourth microphones, the circuit further comprising a summer for combining the outputs of the filters.

14. The system of claim 1, wherein the process signal includes an elevation-dependent spectral notch.

15. The system of claim 1, wherein the hearing protection device includes an ear plug.

16. The system of claim 1, wherein the first and second microphones are mounted on the hearing protection device.

17. The system of claim 1, wherein the first and second microphones are mounted on the outside and near the hearing protection device.

18. A hearing protective system comprising:
a hearing protection device having an inside facing the ear and an outside facing away from the ear;
first and second microphones for receiving a first acoustic signal from an external source, the first and second microphones spaced apart at the outside of the hearing protection device and producing electrical signals based on the first acoustic signal;
a circuit for receiving the electrical signals and processing the signals to produce a processed signal with notch at a desired frequency; and
a driver for receiving the processed signal and transmitting a second acoustic signal with the frequency notch into the inside of the hearing protection device.

19. The system of claim 18, further comprising third and fourth microphones, the microphones being arranged substantially in the shape of a square at the outside of the hearing protection device.

20. The system of claim 18, wherein the hearing protection device includes a muff with a closed design to substantially reduce noise incident on the outer surface.

21. The system of claim 18, wherein there are two and only two microphones per ear.

22. The system of claim 19, wherein there are four and only four microphones per ear.

23. A method for generating directional cues in an audio signal derived from a processor and a microphone array and received by a hearing protection device with first and second spaced microphones, the method comprising:
receiving an acoustic signal with the first and second microphones;
producing respective first and second signals as a representation of the acoustic signals received by the first and second microphones;
filtering and combining the first and second signals in accordance with head-related transfer function data to produce a processed signal; and
providing the processed signal to an inside of the hearing protection device for hearing by a wearer of the protector.

24. The method of claim 23, wherein the processed signal has a frequency notch.

25. The method of claim 23, wherein the filtering includes using an array of HRTFs stored in a memory storage device.

26. The method of claim 23, wherein the hearing protection device includes a muff.

27. The method of claim 23, wherein the hearing protection device includes an ear plug.
